(19) United States
(12) Patent Application Publication
(10) Pub. No.: US 2024/0000366 A1
(43) Pub. Date: Jan. 4, 2024

(54) System and Method for the Acquisition and Display of EEG Data

(71) Applicant: Cadwell Laboratories, Inc., Kennewick, WA (US)

(72) Inventor: John A. Cadwell, Richland, WA (US)

(21) Appl. No.: 18/312,451

(22) Filed: May 4, 2023

Related U.S. Application Data
(60) Provisional application No. 63/364,165, filed on May 4, 2022.

(51) Int. Cl. A61B 5/372; A61B 5/00; A61B 5/384

(52) U.S. Cl. CPC A61B 5/372; A61B 5/384; A61B 5/7264

(57) ABSTRACT

A system for EEG data acquisition and presentation includes a neuromonitoring medical system for capturing electrical activity of a patient's brain as EEG signals via EEG electrodes, a server coupled with the neuromonitoring medical system for processing the captured EEG signals and transmitting the processed EEG signals, and a client device for receiving and displaying the processed EEG signals for viewing by a clinician. The server includes modules for paginating EEG signals into data pages of compressed EEG data, binning the paginated EEG data into groups of predefined frequencies, analyzing the binned EEG data to determine signals of interest, and marking the signals of interest with predefined signal markers. The client device enables the clinician to stop the display upon encountering a signal marker and display windows containing low resolution EEG signals in the vicinity of the EEG data containing the signal marker.

### CROSS-REFERENCE

The present application relies on U.S. Patent Provisional Application No. 63/364,165, titled “System and Method for Acquisition and Display of EEG Data” and filed on May 4, 2022. The above-mentioned application is herein incorporated by reference in its entirety.

### FIELD

The present specification relates to diagnostic systems. In particular, the present specification relates to acquisition, analysis, and presentation of electroencephalography (EEG) data.

### BACKGROUND

Several medical procedures involve using multiple sensors on the human body for the recording and monitoring of data required for patient care. Information, such as vital health parameters, cardiac activity, bio-chemical activity, electrical activity in the brain, gastric activity and physiological data, are usually recorded through on-body or implanted sensors/electrodes which are controlled through a wired or wireless link. Typical patient monitoring systems comprise a control unit connected through a wire to one or more electrodes coupled to the specific body parts of the patient.

Neuromonitoring is the use of electrophysiological methods, such as electroencephalography (EEG), electromyography (EMG), and evoked potentials, to monitor the function of certain neural structures (for example, nerves, spinal cord, brain and muscle). Neuromonitoring is used to evaluate abnormal brain electrical activity, and when applicable, to locate pathologic areas in the brain that can be resected or ablated. Some aspects of neuromonitoring involves placing EEG electrodes on a patient's scalp and within the patient's brain in order to record and monitor the electrical activity from various parts of the patient's brain. EEG procedures are classified as either non-invasive or invasive. In non-invasive EEG, a number of electrodes are deployed on a patient's scalp for recording electrical activity in portions of the underlying brain. In invasive EEG, through surgical intervention, the electrodes are placed directly in or on a sections of the brain, in the form of a strip or grid, or are positioned in the deeper areas of the brain using depth electrodes. Each of these electrodes has multiple contacts wherein each of the multiple contacts is coupled to a wire lead which, in turn, is connected to a control unit adapted to receive and transmit electrical signals. The electrical activity captured by various electrodes is analyzed both visually and by using algorithms, in order to detect any abnormal brain activity and to localize the portion(s) of brain responsible for causing the specific activity/ailment.

The resultant EEG data is very large owing to a high number of electrode recording sites, (which may be more than 200); high sample rates (which may be more than 2000 samples per second); and the long monitoring times (which may range from 3 to 7 days). As a result, data visualization to locate anomalies can be a challenging and time-consuming process. In addition to using detection algorithms to analyze the EEG data for any abnormalities/anomalies, all recorded EEG data is also reviewed visually by an operator/clinician in order to detect either any abnormalities or to recognize trends. The review is often performed in a batch mode, where many hours of pre-recorded EEG data is presented at a high speed, which may be up to 100 times the real time data display speed. The presentation of the data rate during review ranges from 1.05×10⁶ samples per second to 80×10⁶ samples per second. Such a high data rate is not attainable on conventional client-side systems, thereby slowing the actual page display rate to a fraction of the desired display rate. Typical display monitors have a display resolution of approximately 1000-2500 points, providing 100-200 points of resolution per second of displayed data. When the data sample rates exceed about 250 Hz (which is almost always the case) the limited screen resolution makes it impossible to view the high bandwidth EEG data in its entirety. Even 4K high resolution display screens require extreme down-sampling (e.g., 10:1) which obscures high frequency content and reduces the ability of a clinician to detect signals of interest. Further, high frequency EEG signals captured using high sample rates have a lower amplitude than lower frequency EEG signals (ranging from 10-50× smaller) and may have insufficient signal to noise ratios impacting their visibility against background noise.

Data handling techniques such as data caching and data compression are insufficient to transfer such large volumes of data across networks for display at a clinician's workstation, even though screen resolution can be artificially enhanced to some extent. In some cases, a peak-detection method is used for computer analysis of the EEG data. The method comprises measuring the amplitude and time interval between successive maxima (peaks) and minima (troughs) in the EEG signal and includes an amplitude threshold criterion which eliminates the registration of low-voltage activity riding on EEG waves. Conventional EEG analysis methods such as the peak detection method and minimum-maximum data compress enable a clinician to see higher frequency components of EEG signals, however, are still insufficient to enable the clinician to analyze all the captured EEG data.

Various data compression techniques and paging techniques for storage of EEG data in secondary memories are used to provide clinicians with the EEG data for analysis. Most of the energy and waveform morphology is contained in EEG signal components having frequency components below 30 Hz. Highly compressed EEG data loses the small waveform morphology changes during fast compressed data reviews and, as a result, run the risk of a clinician missing or not catching signals of interest from the EEG data. Also, frequently, lossy compressed EEG signals do not match the original EEG signal exactly and, when paging is stopped by a clinician to examine a signal event, the transition from compressed data to full bandwidth data is noticeable. With remote viewing of EEG data such as, but not limited to, over a Citrix® server, page data is not always transferred at the same rate as data updates to the page which may result in some pages being skipped without a clinician viewing the data and/or even being aware of the skipped data.

During an EEG procedure, some high-frequency oscillations (HFOs)—brain waves having a frequency greater than approximately 80 Hz, which may be associated with seizure activity, may also be recorded. Such HFO signals are difficult to detect as the amplitude of these signals is an order of magnitude smaller than the other recorded EEG signals, thus a clinician may not be able to detect HFOs even when a peak detect method is used. Commonly, HFO signals are characterized as signal frequency bursts lasting for approximately 0.1 seconds, and it may be near impossible for a clinician to notice such infrequent bursts while analyzing compressed EEG signals rendered at a high speed. Further, clustering the magnitude of HFO signal bursts over a 24-hour period may lead to anomalous deductions by the clinician. It is often difficult for a clinician to review epileptiform activity in EEG signals—such as spikes, sharp waves, or spike-and-wave complexes
when data is rendered at a high speed. Further, artifact, such as mains interference signals, can obscure smaller EEG signals making it even more difficult to correctly analyze EEG signals.

Hence, there is a need for a system and method for displaying of large volumes of compressed EEG data wherein a clinician viewing the data can quickly notice any signals of interest and request uncompressed versions of the data comprising the signals of interest for a more detailed review.

### SUMMARY

The following embodiments and aspects thereof are described and illustrated in conjunction with systems, tools and methods, which are meant to be exemplary and illustrative, and not limiting in scope. The present application discloses numerous embodiments.

The present specification discloses a system for electroencephalogram (EEG) data acquisition and presentation comprising: a neuromonitoring medical system configured to capture electrical activity of a patient's brain as EEG signals via a plurality of EEG electrodes; a server coupled with the neuromonitoring medical system configured to receive and process the EEG signals wherein the server comprises: a data compression module configured to paginate the EEG signals into a plurality of pages, wherein each of the plurality of pages is a predefined size that comprises compressed EEG data derived from the EEG signals; a data segmentation module configured to segment the paginated, compressed EEG data into groups, wherein each group is defined by a predefined frequency range; a data segment analysis module configured to analyze the EEG data to determine signals corresponding to EEG events; and a client device adapted to receive and display the analyzed EEG data for viewing by a clinician, wherein the client device comprises at least one processor and programmatic instructions that, when executed by the at least one processor: sequentially displays a plurality of windows, wherein each of the plurality of windows is adapted to display a first version of the EEG data having a first resolution and wherein each of the plurality of windows is configured to display a predefined time range of the first version of the EEG data; receives a selection of one or more of the displayed plurality of windows; based on the selection, fetches a second version of the EEG data, wherein the second version has a second resolution and wherein the second resolution is greater than the first resolution; and displays the second version of the EEG data.

Optionally, the second version of the EEG data is an uncompressed version of the EEG data and the first version of the EEG data is a compressed version of the EEG data.

Optionally, the data segment analysis module is configured to analyze the EEG data to determine signals of interest and to mark the signals of interest and the EEG events with signal markers. Optionally, the data segment analysis module is configured to mark the signals of interest using a first type of predefined signal markers and mark the EEG events using a second type of predefined signal markers, wherein the first type of predefined signal markers is different from the second type of predefined signal markers. Optionally, at least some of the signals of interest comprise high frequency oscillations (HFO). Optionally, at least some of the signals of interest comprise one or more EEG signals having a frequency outside of predefined frequency ranges, wherein the ranges include at least 80 Hz to 250 Hz and 250 Hz to 500 Hz. Optionally, the predefined signal markers comprise audible signal markers.

Optionally, the predefined time range displayed by each of the plurality of windows is in a range of 1 second to 5 minutes.

Optionally, each of the plurality of windows is adapted to display 1 to 120 seconds of the first version of the EEG data occurring before one of the predefined signal markers and adapted to display 1 to 120 seconds of the first version of the EEG data occurring after said one of the predefined signal markers.

Optionally, the server further comprises a cache memory wherein the cache memory is configured to store the segmented, paginated, compressed EEG data. Optionally, the server further comprises a cache controller coupled with the cache memory wherein the cache controller is configured to control a transmission of the segmented, paginated, compressed EEG data stored in the cache memory to the client device. Optionally, the cache memory is adapted to store the second version of the EEG data corresponding to the selected one of the plurality of windows.

Optionally, the client device further comprises programmatic instructions that, when executed by the at least one processor, generates a three dimensional display of the first version of the EEG data wherein the three dimensional display comprises signal markers visually indicating where high frequency oscillations occur in the EEG signals.

Optionally, the client device further comprises programmatic instructions that, when executed by the at least one processor, enables a user to stop a display of the first version of the EEG data upon encountering one or more of the signal markers.

Optionally, a first group of compressed EEG data is defined by a frequency ranging 100 Hz to 150 Hz, a second group of compressed EEG data is defined by a frequency ranging from 150 Hz to 200 Hz, and subsequent groups of compressed EEG data are defined by increasing 50 Hz ranges.

The present specification also discloses a method of processing and presenting EEG data comprising: acquiring EEG data; paginating the EEG data into a plurality of pages, wherein each of the plurality of pages has a predefined size and comprises the EEG data in a compressed format; grouping the paginated, compressed EEG data into one or more frequency bins; analyzing the grouped, paginated, compressed EEG data to determine at least one of signals of interest or signals corresponding to EEG events; marking at least one of the signals of interest or the signals corresponding to EEG events with signal markers; displaying the grouped, paginated, compressed EEG data comprising the signal markers by: enabling a user to stop the display upon encountering at least one of the signal markers; displaying a plurality of windows, wherein each of the plurality of windows is adapted to display a low-resolution version of the EEG signals in a vicinity of the grouped, paginated, compressed EEG data containing the signal marker; enabling the user to select at least one of the plurality of windows; and fetching a high-resolution version of the EEG data in the selected at least one of the plurality of windows; and displaying the fetched high-resolution EEG data.

Optionally, the signal markers comprise a first type and a second type, wherein the first type of signal marker is used to mark signals of interest and wherein the second type of signal marker is used to mark signals corresponding to EEG events.

Optionally, a signal of interest comprises high frequency oscillations (HFO).

Optionally, a signal of interest comprises one or more EEG signals having a frequency outside of predefined frequency ranges, wherein the ranges include at least 80 Hz to 250 Hz and 250 Hz to 500 Hz.

Optionally, the signal markers comprise audible signal markers.

Optionally, the method further comprises displaying three dimensional compressed EEG signals comprising high frequency oscillations signal markers superimposed upon other EEG signals.

Optionally, grouping the paginated EEG data into frequency bins comprises groups of EEG signals having a time resolution of approximately 100 msec and having bandwidth spreads in a range of 50 Hz to 100 Hz.

Optionally, each of the EEG events comprises one or more of a stimulus trigger, an EEG response to a stimulus, artifacts of brain activity, and specific patterns of brain activity.

Optionally, displaying the grouped, compressed, paginated EEG data comprises displaying the grouped, compressed, paginated EEG data at a rate ranging from 1 to 100 pages per second.

Optionally, a first bin of compressed EEG data is defined by a frequency ranging 100 Hz to 150 Hz, a second bin of compressed EEG data is defined by a frequency ranging from 150 Hz to 200 Hz, and subsequent bins of compressed EEG data are defined by increasing 50 Hz ranges.

The present specification also discloses a system for electroencephalogram (EEG) data acquisition and presentation comprising: an EEG data acquisition system configured to capture an electrical activity of a patient's brain as EEG signals via a plurality of EEG electrodes; a server coupled with the neuromonitoring medical system configured to receive and process the EEG signals wherein the server comprises: a data compression module configured to paginate the EEG signals into a plurality of pages, wherein each of the plurality of pages comprises compressed EEG data derived from the EEG signals; a data segmentation module configured to segment the paginated, compressed EEG data into groups, wherein each group is defined by a predefined frequency range; a data segment analysis module configured to analyze the EEG data to determine data corresponding to EEG events; and a client device adapted to receive and display the compressed and analyzed EEG data for viewing by a clinician, wherein the client device comprises at least one processor and programmatic instructions that, when executed by the at least one processor: sequentially displays a plurality of windows, wherein each of the plurality of windows is adapted to display a first version of the EEG data having a first resolution and display one or more first parameters and wherein each of the plurality of windows is configured to display a predefined time range of the first version of the EEG data; receives a first selection of one or more of the displayed plurality of windows; based on the first selection, fetches a second version of the EEG data, wherein the second version has a second resolution and is associated with one or more second parameters and wherein the second resolution is greater than the first resolution and at least one of the second parameters is different from at least one of the first parameters; displays the second version of the EEG data; receives a second selection of the one or more of the displayed plurality of windows chosen by the first selection; based on the second selection, fetches a third version of the EEG data, wherein the third version has the second resolution and is associated with at least one of the first parameters; and displays the third version of the EEG data.

Optionally, the first parameters and the second parameters comprise at least one of high cut filter settings, low cut filter settings, gain, sweep, or page position.

Optionally, the client device further comprises programmatic instructions that, when executed, generates a displayable navigation bar having a first portion and a second portion and that is adapted to cause the first selection when the first portion is activated. Optionally, the displayable navigation bar is further adapted to cause the selection when the second portion is activated.

Optionally, the client device further comprises programmatic instructions that, when executed by the at least one processor, generates a three dimensional display of the compressed and analyzed EEG data wherein the three dimensional display comprises signal markers visually indicating where high frequency oscillations occur in the EEG signals.

Optionally, the client device further comprises programmatic instructions that, when executed by the at least one processor, are configured to stop a display of the compressed and analyzed EEG data upon encountering one or more of the signal markers.

In embodiments, the present specification also discloses a system for EEG data acquisition and presentation comprising: a neuromonitoring medical system configured to capture electrical activity of a patient's brain as electroencephalogram (EEG) signals via a plurality of EEG electrodes; a server coupled with the neuromonitoring medical system configured to receive and process the EEG signals and to transmit the processed EEG signals, the server comprising: a data compression module configured to paginate the EEG signals into a plurality of pages, wherein each of the plurality of pages is a predefined size that comprises compressed EEG data from the EEG signals; a data segmentation module configured to segment the paginated, compressed EEG data into groups of predefined frequencies; a data segment analysis module configured to analyze the clustered EEG data to determine signals of interest, to determine signals corresponding to EEG events, and to mark the signals of interest and the signals corresponding to EEG events with predefined signal markers; and a client device for receiving and displaying the analyzed EEG data for viewing by a clinician, wherein the client device is configured to: enable the clinician to control the display of the analyzed EEG data; display a plurality of windows, wherein each of the plurality of windows is adapted to display a low-resolution version of the EEG data containing the signal marker and containing the EEG in a time range 60 seconds before the signal marker and in a time range 60 seconds after the signal marker; enable the clinician to select one or more of the displayed windows; fetch an uncompressed version of the low-resolution version of the EEG data corresponding to the one or more selected windows; and display the uncompressed EEG data.

Optionally, the data server further comprises a cache memory, configured to store the clustered paginated EEG data comprising signal markers. Optionally, the system further comprises a cache controller coupled with the cache memory configured to control a transmission of the clustered paginated EEG data stored in the cache memory to the client device. Optionally, the cache memory is adapted to store uncompressed EEG data corresponding to one or more windows selected by the clinician.

Optionally, a first type of predefined signal markers are used to mark signals of interest and a second type of predefined signal markers different from the first type are used to mark signals corresponding to EEG events. Optionally, the signal markers comprise predefined colors and alphanumeric characters. Optionally, the signal markers comprise audible signal markers.

Optionally, a signal of interest comprises high frequency oscillations (HFO). Optionally, a signal of interest comprises one or more EEG signals having a frequency outside of a predefined frequency range.

Optionally, the client device displays a three dimensional display of compressed EEG signals comprising high frequency oscillations signal markers superimposed upon other EEG signals. Optionally, the client device is configured to enable the clinician to stop the display of the analyzed EEG data upon encountering one or more of the signal markers.

In some embodiments, the present specification discloses a method of processing and presenting EEG data comprising: acquiring EEG data; paginating the EEG data into a plurality of pages, wherein each of the plurality of pages has a predefined size and comprises the EEG data in a compressed format; clustering the paginated, compressed EEG data into groups of predefined frequencies; analyzing the clustered, paginated, compressed EEG data to determine signals of interest and signals corresponding to EEG events; marking at least one of the signals of interest or the signals corresponding to EEG events with predefined signal markers; displaying the clustered, paginated, compressed EEG data comprising the signal markers for viewing by a clinician by a) enabling the clinician to stop the display upon encountering a signal marker, b) displaying a plurality of windows, wherein each of the plurality of windows displays a low resolution version of the EEG signals in the vicinity of the clustered, paginated, compressed EEG data containing the signal marker or containing the clustered, paginated, compressed EEG data having the signal marker, c) enabling the clinician to select one or more of the displayed windows, d) fetch an uncompressed version of the EEG data in the one or more selected windows and e) display the uncompressed EEG data.

Optionally, wherein a first type of predefined signal markers are used to mark signals of interest and a second type of predefined signal markers different from the first type are used to mark signals corresponding to EEG events. Optionally, the signal markers comprise predefined colors and alphanumeric characters. Optionally, the signal markers comprise audible signal markers.

Optionally, a signal of interest comprises high frequency oscillations (HFO). Optionally, a signal of interest comprises one or more EEG signals having a frequency outside of a predefined frequency range.

Optionally, the method comprises displaying three-dimensional compressed EEG signals comprising high frequency oscillations signal markers superimposed upon other EEG signals.

Optionally, clustering the paginated EEG data into clusters of predefined frequencies comprises creating a data groups of EEG signals having a time resolution of approximately 100 msec, and preferably with bandwidth spreads of 50 Hz to 100 Hz, or any increment therein. Optionally, displaying the clustered, compressed paginated EEG data comprises displaying the clustered, compressed paginated EEG data at a rate of 20 pages per second.

Optionally, each of the EEG events comprises one or more of: a provision of stimulus triggers and an EEG response to said stimulus, artifacts of brain activity, or specific patterns of brain activity.

The aforementioned and other embodiments of the present specification shall be described in greater depth in the drawings and detailed description provided below.

### BRIEF DESCRIPTION OF THE DRAWINGS

The accompanying drawings illustrate various embodiments of systems, methods, and embodiments of various other aspects of the disclosure. Any person with ordinary skill in the art will appreciate that the illustrated element boundaries (e.g. boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1A is a block diagram illustrating a system for EEG data acquisition and presentation, in accordance with an embodiment of the present specification;

FIG. 1B is an illustration of a graphical user interface window displaying fast-paginated EEG data and a plurality of low resolution windows of EEG data enabling a clinician to select a window of interest, in accordance with an embodiment of the present specification;

FIG. 1C illustrates a plurality of modes of operation of the system for EEG data acquisition and presentation, in accordance with an embodiment of the present specification;

FIG. 1D illustrates a plurality of electrodes attached at different locations on a human head for stimulating/recording brain activity, in accordance with an embodiment of the present specification;

FIG. 2 is a block diagram illustrating a process of EEG data acquisition and poly-band sampling, in accordance with an embodiment of the present specification;

FIG. 3 is a pictorial representation of a polyband analysis of filtered EEG signals as shown in FIG. 2;

FIG. 4A is a flowchart illustrating a method for down-sampling and compressing a band of low frequency EEG signals, in accordance with an embodiment of the present specification;

FIG. 4B is a diagram showing an EEG signal sampled at a rate of 2000 samples per second;

FIG. 4C is a diagram showing the signal of FIG. 4B down-sampled and compressed to a rate of 250 samples per second, in accordance with an embodiment of the present specification;

FIG. 5A illustrates a plurality of electrodes attached at different locations in a human brain for stimulating/recording brain activity with the presence of high frequency data shown in color, in accordance with an embodiment of the present specification;

FIG. 5B is an EEG display obtained via the electrodes shown in FIG. 5A, with the presence of high frequency data shown in color, in accordance with an embodiment of the present specification;

FIG. 5C illustrates the EEG display obtained via the electrodes shown in FIG. 5A with high frequency data shown as a frequency shifted burst in accordance with an embodiment of the present specification; and

FIG. 6 is a flowchart detailing a method for acquiring and presenting high frequency EEG data, in accordance with an embodiment of the present specification.

### DETAILED DESCRIPTION

The present specification provides a system and method for compressing high sample rate EEG data and subsequently displaying the EEG data such that it can be rapidly paged. In an embodiment, the present specification also provides a method for marking signals of interest in the EEG data. The marked signals comprise high-frequency oscillation (HFO) signals of interest and/or conventional EEG events. In other embodiments, the present specification provides a method for enabling a clinician viewing the rapidly paginated high sample rate EEG data to see the marked signals, and upon receiving a request from the clinician, the present system is configured to display uncompressed high sample rate EEG signals corresponding to the marked signals for viewing by the clinician. In embodiments, compressed EEG signals and high sample rate EEG signals are displayed without superposition in order to mask the visual transition from one data set to the other.

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

In the description and claims of the application, each of the words “comprise”, “include”, “have”, “contain”, and forms thereof, are not necessarily limited to members in a list with which the words may be associated. Thus, they are intended to be equivalent in meaning and be open-ended in that an item or items following any one of these words is not meant to be an exhaustive listing of such item or items, or meant to be limited to only the listed item or items. It should be noted herein that any feature or component described in association with a specific embodiment may be used and implemented with any other embodiment unless clearly indicated otherwise.

It should also be noted that as used herein and in the appended claims, the singular forms “a,” “an,” and “the” include plural references unless the context dictates otherwise. Although any systems and methods similar or equivalent to those described herein can be used in the practice or testing of embodiments of the present disclosure, the preferred, systems and methods are now described.

In embodiments, the systems and method of the present specification include at least one processor (not shown) to control the operation of the entire system and its components. It should further be appreciated that the at least one processor is capable of processing programmatic instructions, has a memory capable of storing programmatic instructions, and employs software comprised of a plurality of programmatic instructions for performing the processes described herein. In one embodiment, the at least one processor is a computing device capable of receiving, executing, and transmitting a plurality of programmatic instructions stored on a volatile or non-volatile computer readable medium. In addition, the software comprised of a plurality of programmatic instructions for performing the processes described herein may be implemented by a computer processor capable of processing programmatic instructions and a memory capable of storing programmatic instructions. In embodiments, “computing device” is at least one of a cellular phone, PDA, smart phone, tablet computing device, patient monitor, custom kiosk, or other computing device capable of executing programmatic instructions. It should further be appreciated that each device and monitoring system may have wireless and wired receivers and transmitters capable of receiving and transmitting data. Each “computing device” may be coupled to at least one display, which displays information about the patient parameters and the functioning of the system, by means of a graphical user interface (GUI). The GUI also presents various menus that allow users to configure settings according to their requirements.

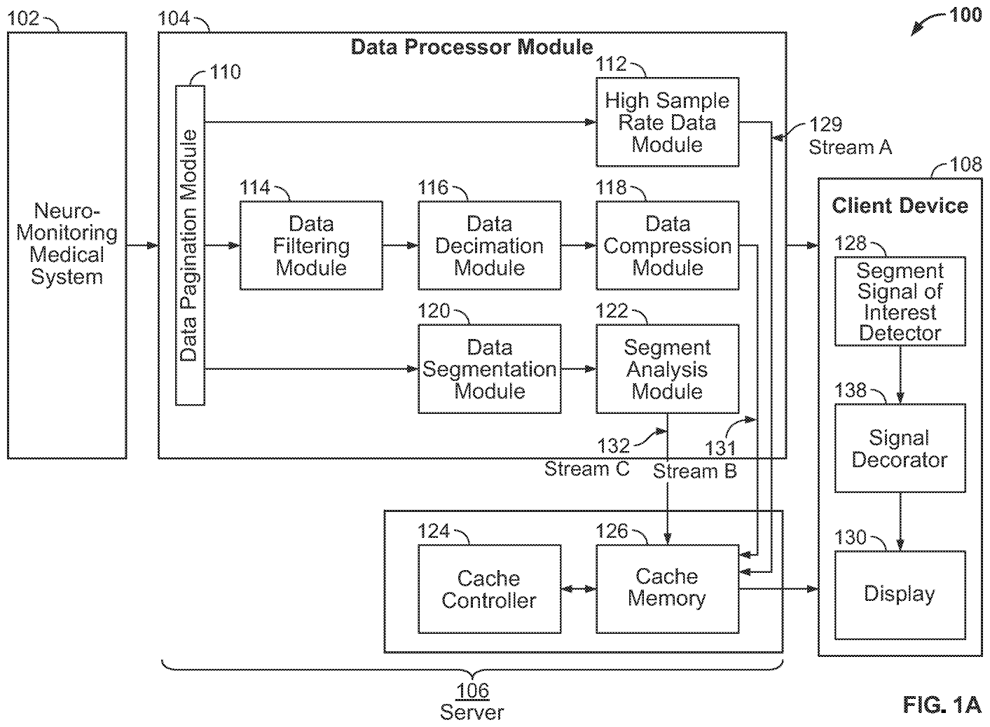

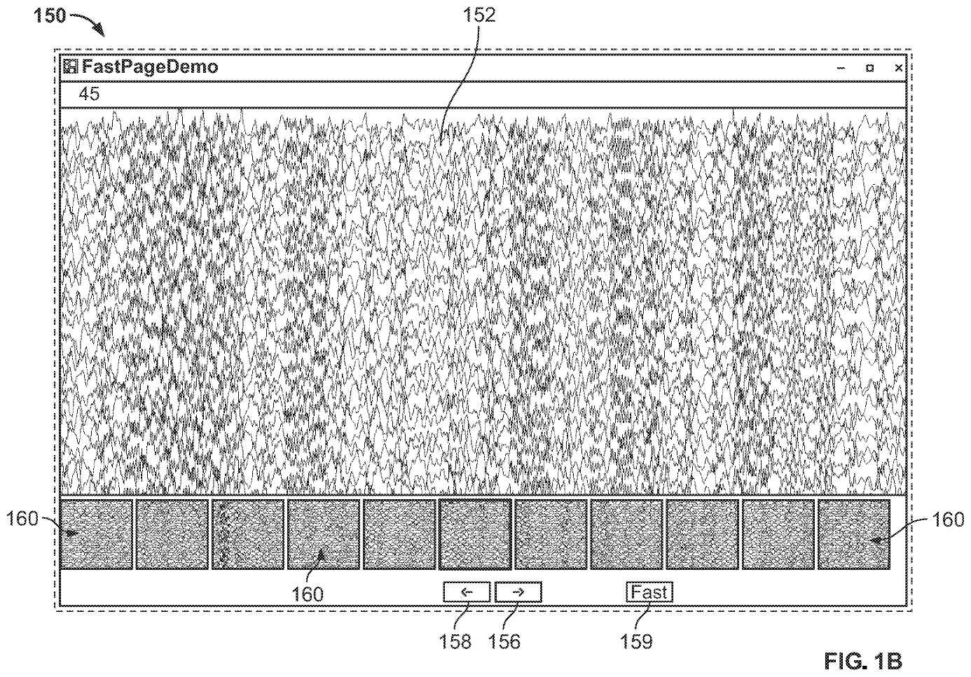

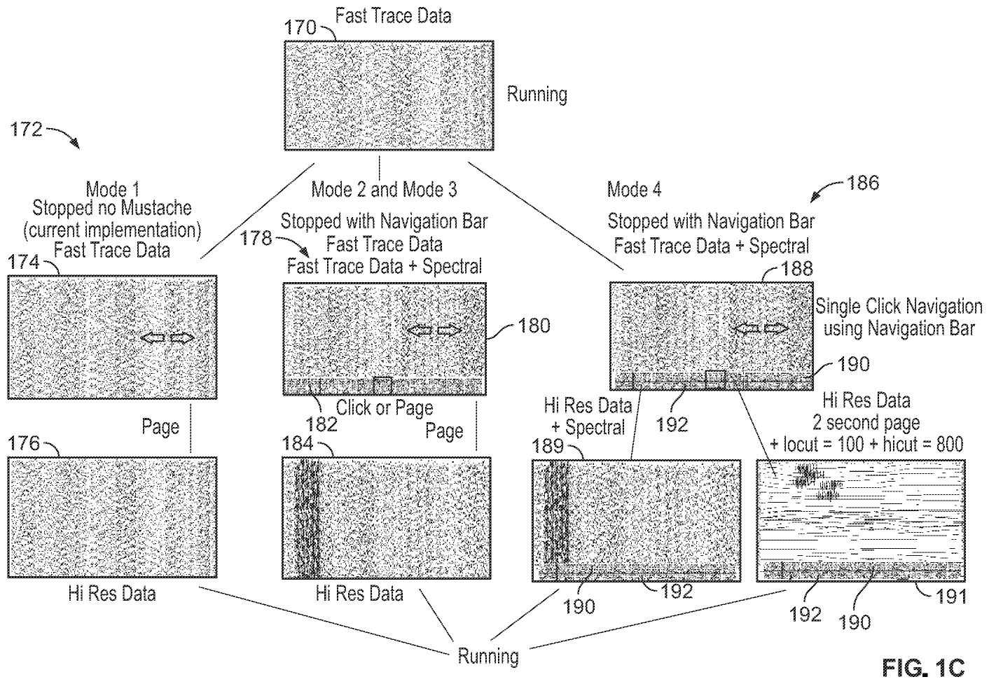

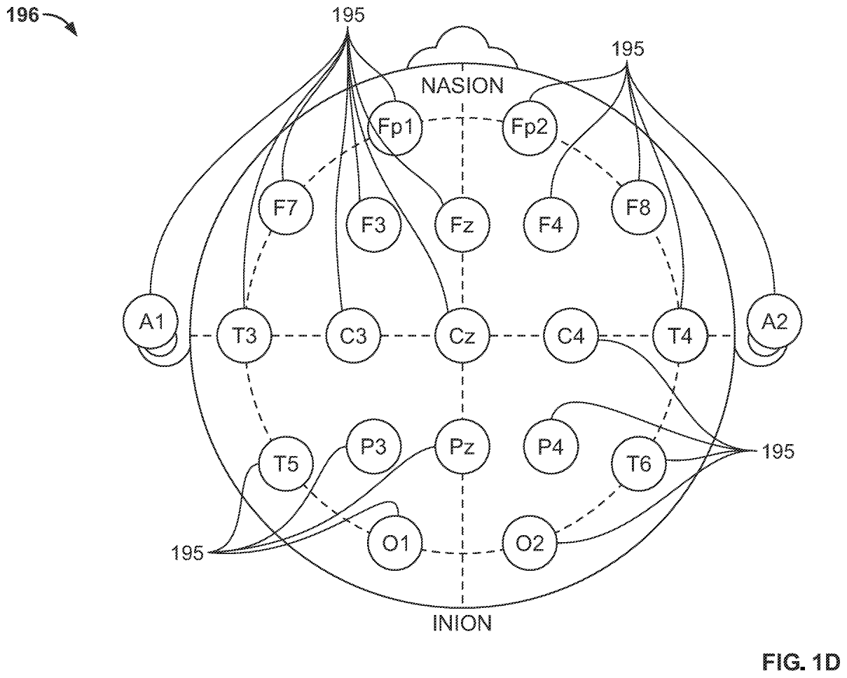

FIG. 1A illustrates a system for EEG data acquisition and presentation, in accordance with an embodiment of the present specification. System 100 comprises a neuromonitoring medical system 102, a server 106 including a data processor module 104, and a client device 108. In embodiments, the neuromonitoring medical system 102 is an EEG (electroencephalography) system that includes a plurality of electrodes and is used and configured for monitoring the neurological state of a patient for diagnosis and preventive treatment of certain diseases and/or for monitoring patients during anesthesia, among other procedures.

During use, the neuromonitoring medical system 102 may be coupled to a patient through a plurality of electrical leads such that each of the leads is coupled to an electrode (not shown) positioned at an appropriate location on the body of the patient, such as, but not limited to, the twenty-one locations on a human head shown in FIG. 1D. FIG. 1D illustrates a plurality of electrodes 195 attached at different locations on a human head 196 for stimulating/recording brain activity, in accordance with an embodiment of the present specification. Each electrode 195 is positioned at a different location to capture the electrical activity in its vicinity, and the input recorded from each electrode 195 is collected as raw EEG data which is transmitted to the data processor module 104. The data processor module 104 comprises a data pagination module 110, a high sample rate module 112, a data filtering module 114, a data decimation module 116, a data compression module 118, a data segmentation module 120, and a segment analysis module 122. In embodiments, the raw EEG data transmitted to and processed by the data processor module 104 is displayed in real time on the client device 108 and stored on the data server 106 for deferred display on the client device 108. In embodiments, the data server 106 comprises a cache memory 126 coupled with a cache controller 124. In embodiments, the client device 108 comprises a segment signal of interest detector 128, a signal decorator 138, and a display 130.

In embodiments, the data processor module 104 is configured to provide at least three data streams obtained by processing the raw EEG data collected by the neuromonitoring medical system 102 and transferred to the data processor module 104 by the system 102. The high sample data rate module 112 is configured to generate a high sample rate data stream 129 (Stream A) at a specified storage rate. Data compression module 118 is configured to generate a compressed data stream 131 (Stream B) at a fractional rate ranging from ⅛ to ⅓ of the raw EEG data sample rate. The segment analysis module 122 is configured to generate a segmental band data stream 132 (Stream C). In embodiments, storage rates of the generated data streams A, B and C are approximately 500 to 200 samples per second, 250 samples per second and 10 samples per second per frequency band, respectively.

The data pagination module 110 is configured to paginate raw EEG data obtained from the neuromonitoring medical system 102 into smaller portions of data. As is known, pagination is a technique used to divide a data set into smaller, more manageable chunks, often in the form of ‘pages’ of data that is presented one ‘page’ at a time to a client device. In a preferred embodiment, the pagination size provided by the system 100 is one second. Thus, in embodiments, the data is stored and analyzed in segments having a duration of 1 second. A displayed page consists of multiple 1 second paginated pages. The pages are then subdivided into nominal 0.1 second segments for analysis, as described below.

The displayed data can range from 1 second to several minutes, for example, 5 minutes, but preferably is often in the range of 10-17 seconds per ‘page’ and more preferably 17 seconds which, on a wide monitor, will maintain the aspect ratio of the displayed data. In an embodiment, the high sample rate module 112 is configured to convert the paginated data obtained from the data pagination module 110 into storage ready data at a predefined high sample rate data stream 129 (Stream A). In an embodiment, the paginated data is sequentially processed by a data filtering module 114, a data decimation module 116, and a data compression module 118 which are configured to generate a compressed data stream 131 (compressed waveform format, Stream B). In embodiments, the data filtering module 114 is configured to remove signal content which has little value and is harder to compress (typically on the order of 50 Hz to 60 Hz). The data decimation module 116 is configured, in embodiments, to reduce the sample count by eliminating oversampled data points, using an algorithm that is known to one of ordinary skill in the art. In an embodiment, said algorithm, for example, applies filters such as, a notch filter at 50 Hz or 60 Hz and a high cut filter at 70 Hz to the paginated data, and reduces sample rate of the data by an by integral ratio (typically 2, 4, 8 or 16 to 1) to approximately 250 samples per second.

In an embodiment, the paginated data is processed by a data segmentation module 120 which is configured to generate a segmental band data stream 132 (Stream C). The segment count may be based on the time and frequency resolution needed for representing high frequency events. In a preferred implementation a time resolution in a range of 5 msec to 1000 msec, or any increment therein, and more preferably approximately 100 msec, is used. As is known to those skilled in the art, shorter data segments have better time resolution and longer data segments have better frequency resolution.

The data segmentation module 120 is configured to form clusters of the paginated data by using a predefined clustering rule. In an embodiment, the paginated data is bucketed into data groups (sets of polyband values) each having a time resolution of approximately 100 msec. The segment analysis module 122 is configured to analyze the data segments/buckets. The segment analysis module 122 is configured to measure and record the amplitude of each of the several frequency bands in the paginated data, preferably with bandwidths whose range increases with frequency, from 50 Hz to 150 Hz per band and spanning the entire range from 0 Hz to the Nyquist frequency. The band frequencies are chosen to provide multiple adjacent bands centered around known signals of interest. As is known, EEG activity of a person, at low frequencies, is characterized by waves in four bands known as delta, theta, alpha and beta. In embodiments, it is advantageous to store a total signal energy at all frequencies grouped in energy bands instead of storing a voltage value for each individual frequency, as this reduces data rates by a large factor. In an exemplary embodiment, data rate is reduced by a factor of approximately 20:1. In embodiments, the energy levels per band are used to annotate or simulate the signals of interest in a manner that is easily visualized. In an embodiment, paginated data signals having a frequency ranging from 100 Hz to 150 Hz are clustered into one bucket/group, paginated data signals having a frequency ranging from 150 Hz to 200 Hz are clustered in another bucket/group, and so on such that subsequent buckets/groups are defined by increasing 50 Hz ranges. In an embodiment, paginated data signals having a frequency of either 50 Hz or 60 Hz, based on the system main frequency, are clustered into one bucket/group and are used to reinsert the mains artifact into the displayed data signals (such as EEG waveform) for quality assessment.

The segment signal of interest detector 128 is configured to analyze the grouped/clustered data to find signals of interest, which are then flagged. In an embodiment, the client device 108 comprises the segment signal of interest detector 128. However, in other embodiments, the segment signal of interest detector 128 may also be implemented in other components of the system 100, may generate events in addition to those described here, and may operate in the absence of a display. In embodiments, signals of interest may be signals having amplitudes lying outside a predefined range, such as high frequency oscillation (HFO) EEG signals which are grouped into frequency bands ranging from 80 Hz to 250 Hz (ripples) and 250 Hz to 500 Hz (fast ripples). In an embodiment, physiological signals or artifacts such as, but not limited to eye movements, heartbeats, patient movements stimulus/stimulation triggers, or a patient's reaction/response to said stimulus, may appear as signals of interest which are then treated as false positives. In some embodiments, such physiological signals or artifacts may have frequencies overlapping the frequency ranges of HFO signals, however said signals do not match all the predetermined criteria for being classified as HFO signals. In embodiments, determination of signals of interest is based not only on signal frequency, but also on a location and electrical field of the signal.

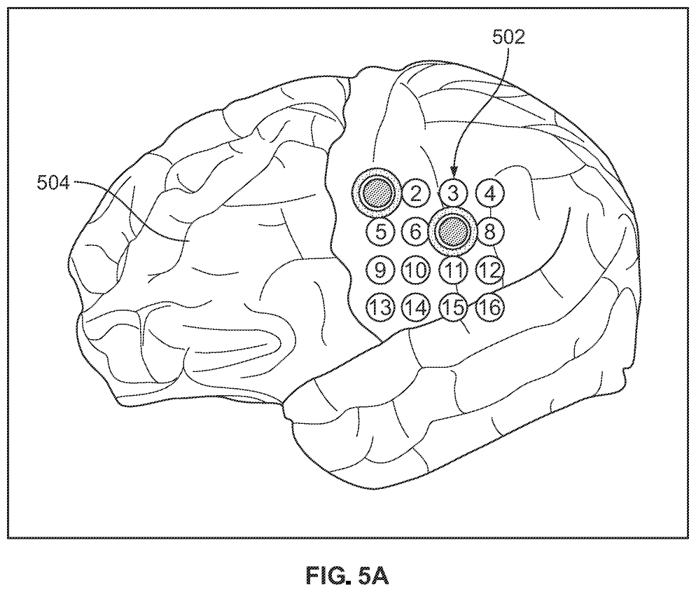

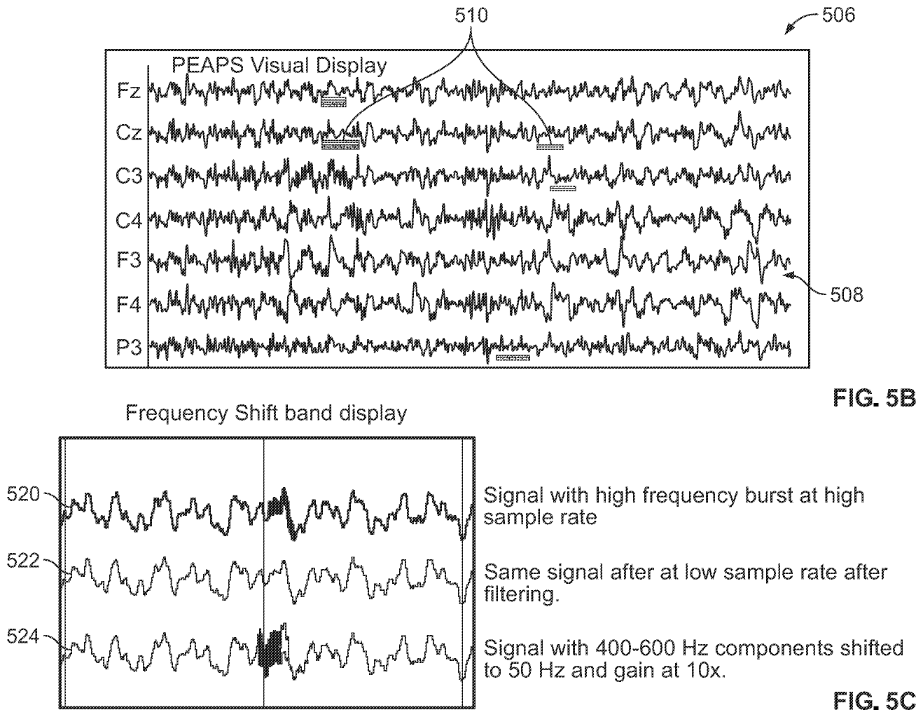

It should be noted that in embodiments, conventional events are often displayed textually on the EEG page or over a specific trace. In embodiments, the signals of interest, specifically HFOs, correspond to the signals within the clustered data that are generated by frequencies lying in a range not clearly visible on a display 130 of the client device 108. In embodiments, the signals of interest are displayed at the time and over the trace (original EEG signal) from which said signals of interest were generated. In embodiments, a plurality of display implementations may be used (FIGS. 5A-5C) for displaying the signals of interest. In some embodiments, the signals of interest may be marked with predefined colors. In an embodiment, the signals of interest may be used to generate frequency shifted (and thus displayable) signals added to the EEG waveform obtained by the neuromonitoring medical system 102, by using the signal decorator 138. In embodiments, the signal decorator 138 is used to highlight, accentuate or locate the signals of interest within the EEG waveform by adding synthesized frequency shifted waves to the waveform. In embodiments, the signal decorator 138 accentuates signals of interest by super positioning said signals as colored waveforms, or by changing background display colors. The presence and characteristics of signals of interest are determined by the presence of energy in the high frequency bands.

In embodiments, the high sample rate data stream 129 (stream A), the compressed data stream 131 (Stream B), and the segmental band data stream 132 (Stream C) are stored in the cache memory 126 of the data server 106 and are transmitted to the client device 108. In an embodiment, the cache controller 124 is configured to transmit the data stored in the cache memory 126 to the client device 108. Since compressed data is transmitted to the client device 108, less bandwidth is utilized for transmission which improves performance on bandwidth limited networks. The client device 108 enables fast review of the transmitted data, during which the client device 108 automatically advances through displayed pages at a fast predefined rate, such as, but not limited to 15 pages per second. A clinician viewing the compressed data displayed on the display 130 of the client device 108 may stop fast review when encountering event markers or signal of interest markers. Upon stopping fast review, a number of preceding pages (selected as a number of pages that is large enough to provide a “small” enough window to see the event, while maintaining an aspect ratio of the EEG display), for example 10 to 15 pages, are displayed on the display 130 in multiple small windows (as shown in FIG. 1B), allowing the clinician to select a page of interest. When fast review stops and/or when the clinician selects one of the displayed windows, the entirety of the selected high sample rate page data is streamed from the server 106 to the display 130 of the client device 108. This allows compressed EEG data to be delivered quickly to the client device 108, wherein only the data of interest that the clinician wishes to view and has selected from among the multiple small windows is displayed in full resolution. In some embodiments, each window is adapted to display 5 to 30 seconds of the first version of the EEG data occurring before one of the predefined signal markers. A total time for which the EEG data is displayed is typically 120 seconds, which ensures that all signals of interest are displayed. In embodiments, a clinician's reaction time (i.e. the time taken by the clinician to stop fast pagination after the clinician notices a signal of interest or an EEG event on the display screen) is approximately 0.7 seconds, and data pages shown for said duration before pagination is stopped are available for selection by the clinician. At faster paging speeds, the clinician's reaction time increases and data for a longer preceding time is required to be displayed, which may be in the form of a single very long page or multiple shorter pages (as shown in FIG. 1B).

In an embodiment, the system 100 is configured to provide a ‘page select’ functionality to the clinician viewing the EEG data via the client device 108, for minimizing disparities between full resolution EEG data made available to the client device 108 when the clinician stops pagination, and fast paginated EEG data. When a clinician viewing the fast paginated data displayed on the client device 108 stops pagination through any input known to persons of ordinary skill in the art upon encountering event markers or signal of interest markers, in most cases, the page of interest is several pages back/before the displayed page at which the clinician terminates pagination, due to the fast speed of the page scroll and latencies in human reaction time.

FIG. 1B illustrates a window displaying fast paginated EEG data and a plurality of low-resolution windows of EEG data enabling a clinician to select a window of interest, in accordance with an embodiment of the present specification. In an embodiment, 11 low resolution windows of EEG data are displayed for approximately 10 seconds each. Display 150 illustrates fast paginated EEG data 152 and a plurality of windows 160 displaying low resolution EEG data lying in the vicinity of the EEG data 152 at which a clinician viewing the data stops data pagination. The low resolution EEG data comprises a first version of EEG data having a first resolution. Data windows 160 may be referred to as ‘satellite views’ of fast data viewed before the data 152. This satellite view method allows the clinician to select a page comprising EEG data of interest. Once the clinician selects any of the windows 160, the EEG data of the selected window is displayed on the screen in full resolution (uncompressed). The full resolution (uncompressed) EEG data comprise a second version of EEG data having a second resolution, wherein the second resolution is greater than the first resolution. Hence, only when the clinician selects a particular window, is the EEG data corresponding to that window retrieved from the server (which stores both compressed and uncompressed EEG data) to the client device of the clinician. In an embodiment, the fast paginated data 152 being viewed by the clinician when the clinician stops pagination is not shown in any of the low-resolution data windows 160, as in most cases, the page of interest is several pages back/before the displayed page at which the clinician terminate pagination, due to the fast speed of the page scroll and latencies in human reaction time. Buttons 156, 158 adapted to cause a forward and backward movement, respectively, to view fast EEG data are provided on the display 150. A button 159 allows the clinician to resume viewing fast EEG data after having viewed a full resolution data of interest.

Conventionally, full resolution EEG data obtained from 200 channels and sampled for each channel at a rate of 2000 samples/second is displayed at approximately 3 pages per second, when manually advanced, for viewing on a client device. Referring to FIG. 1A, in an embodiment, the system of the present specification displays fast paginated data on the client device 108 at a rate of approximately 20 pages per second. In various embodiments, the system displays fast paginated data on the client device 108 at a rate ranging from 1 to 100 pages per second. In an embodiment, the cache controller 124 is configured to enable switching between transmission of fast EEG data and full resolution EEG from the cache memory 126 based on a paging status. When the clinician viewing the fast paginated EEG data on the client device 108 stops pagination and requests a selected page of high-resolution data, subsequent pages of high-resolution data are stored in the cache memory 126.

In an embodiment, the clinician's reaction time (i.e. the time taken by the clinician to stop fast pagination after the clinician notices a signal of interest or an EEG event on the display screen) is determined by tracking the number of the previously displayed page showing the signal of interest of the EEG event most often selected by the clinician after stopping pagination. For example, if pagination stops at a page number 100, and the page selected by the clinician from the satellite view is page 96 or page 97, the system records this information, uses it to determine the clinician's reaction time for noticing signals of interest and stopping pagination, and subsequently uses that reaction time to automatically select, or recommend the selection of, the page of interest based on the point of stopped pagination.

FIG. 1C illustrates a plurality of modes of operation of the system for EEG data acquisition and presentation, in accordance with an embodiment of the present specification. As shown, fast paginated EEG data is displayed in window 170. In a first mode 172 of operation, fast paginated data page generated by using a logic less template engine is displayed in window 174, which, upon being clicked, displays the corresponding high-resolution data in window 176. As is known to persons of ordinary skill in the art, a logic less engine is a set of programmatic instructions, which may be compiled as a single executable software application or multiple separate modules, that comprises tag names backed by a model object referencing data, such as high-resolution EEG data, for a template and that does not have logic to support if-else statements or loops. An exemplary logic less template engine is referred to as “Mustache” because the tag names are surrounded by the following symbols: {{ }}.

In second and third modes 178 of operation, a display 180 illustrates fast paginated EEG data and a navigation bar 182, wherein the display 180 shows low resolution EEG data lying in the vicinity of the EEG where a clinician viewing the data has stopped data pagination using the navigation bar 182. Once the clinician has stopped navigation and clicks in the window 180, the EEG data is displayed in window 184 in full resolution (uncompressed). Referring to the second and third modes 178, if a clinician wishes to view different parameters in high resolution data display 184, such as, high cut and low cut filter settings, gain, sweep and page position, these parameters are preferably changed manually. These parameters may then be changed back manually when returning to the parameters originally displayed in high resolution display 184.

In a fourth mode 186 of operation, which depicts a single click navigation mode, a display 188 illustrates fast paginated EEG data, a dual mode selector 190 and a revert bar 192. In some embodiments, the dual mode selector 190 and revert bar 192, which can be any shaped icon, are color coded to assist the clinician in selecting the appropriate bar in order to reduce the time of the clinician viewing the record. For example, in some embodiments, the dual mode selector 190 is orange and the revert bar 192 is green. Clicking the dual mode selector 190 allows viewing of any sub-segment of any page displayed in display 188 in a high resolution mode, such as display 191, with automatic changing of the parameters. For example, clicking dual mode selector 190 in display 188 automatically changes the viewing parameters to show a high resolution data 2 second page with a low cut (locut) filter equal to 100 Hz and a high cut (hicut) filter equal to 800 Hz in display 191. Clicking the revert bar 192 reverts the sub-segment display 191 to a high resolution display with the parameters changed back to the original data parameters automatically, as shown in display 189. The single click navigation mode 186 significantly reduces the clinician's click count and time spent reading the displayed EEG data. In various embodiments, said single click navigation mode may be implemented by using any suitable user interface and is not limited to the use of the dual mode selector 190 and revert bar 192 shown in FIG. 1C.

Referring back to FIG. 1A, the determined reaction time is used by the system 100 to determine which pages to load in the cache memory 126 for rapid transmission to the client device 108. Subsequently, when the clinician stops pagination, the most likely page and its immediate predecessor and successor, based on the previously determined clinician's reaction time, are then automatically loaded into the cache memory 126 for rapid transmission and rendering on the display 130. In an embodiment, a new page/section of data requested by the clinician may take up to 300 msec to load onto the client device 108. During fast EEG data review on the client device 108, only fast EEG data is cached in the cache memory 126 and the cached pages comprise a predefined number of seconds of future EEG data. In an embodiment, more than 100 seconds of future EEG data is cached in the cache memory 126. The cached data which has been displayed is retained to accommodate satellite view display before being discarded. In an embodiment, data display rates on the client device 108 ranges up to 15 pages per second. In embodiments, the data display rate may change dynamically if the cache memory 126 cannot be maintained at the specified page rate.

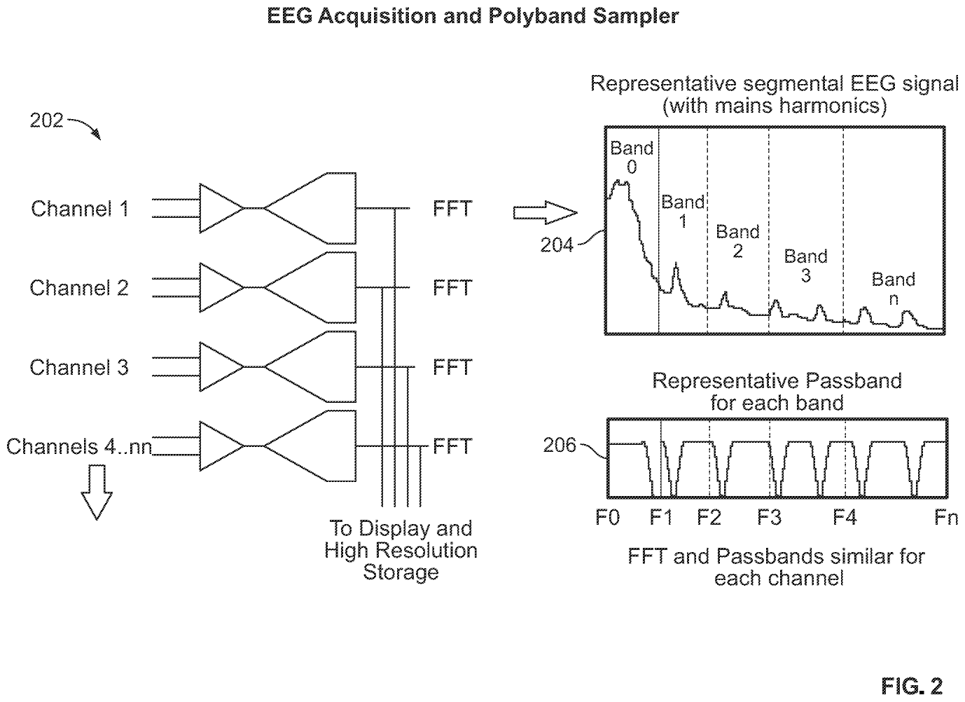

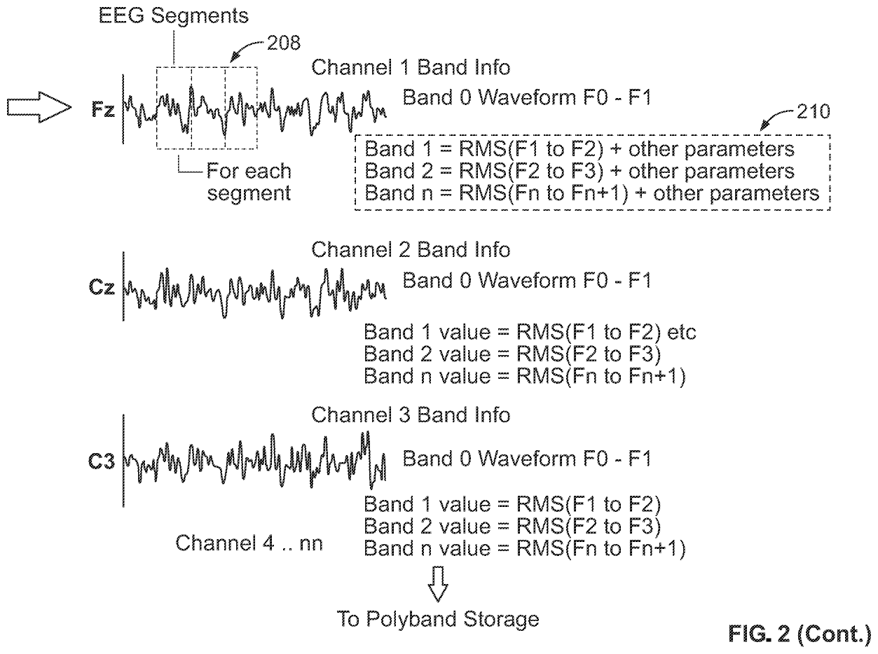

FIG. 2 illustrates a process of EEG data acquisition and poly-band sampling, in accordance with an embodiment of the present specification. Section 202 illustrates EEG data acquired from a plurality of channels, wherein each channel receives signal/data from an electrical lead coupled to an electrode positioned at an appropriate location on the body of a patient to capture the electrical activity in its vicinity. Each channel corresponds to a different electrode. In embodiments, the signals received from the channels are amplified, digitized, and convoluted using data processing algorithms, such as, but not limited to fast Fourier transforms (FFT). In embodiments, FFT is used to change time domain signals received from the channels into frequency domain signals. Graph 204 depicts the frequency domain representation of the EEG signal (containing mains harmonics at 60 Hz and its multiples at 120, 180, etc.) for one data segment 208 bucketed into predefined frequency bands. In an embodiment 8 bands are used with frequencies ranging from 30-50 Hz, 80-125 Hz, 125-175 Hz, 175-250 Hz, 250-350 Hz, 350-450 Hz, 450-600 Hz, and 600-800 Hz. In embodiments, graph 206 depicts a pre-filtering passband used to remove non-physiologic noise, such as, but not limited to, mains interference from data segment 208. The FFT and passband filtering process for each of the data segments for each of the channels, such as, for example, channel ‘1’: data segments ‘0’ to ‘m’, to channel ‘n’: data segments ‘0’ to ‘m’, is similar to that shown in graph 206 of FIG. 2. In an embodiment, a mean voltage of the signals in each frequency bucket is computed using a root mean square (RMS) calculation as is dispersion, using standard deviation, and peak frequency calculation. Each segmental poly-band 210 thus provides a parametric description of the original signal for that data segment. In various embodiments, other algorithms generating other parametric values for the data segments may be used, and some of the parameters shown in FIG. 2 may be omitted, or other parameters may be added without changing the intent and scope of the present specification. In an embodiment, exemplary parameters comprise, but are not limited to: a scale factor for each channel computed by a compression algorithm; and mean, standard deviation or other statistical value of the data in a frequency band. In embodiments, as shown in FIG. 2: band 0 waveform is depicted as ‘F0-F1’; each band 1 waveform comprises the RMS voltage of the FFT spectrum from F1 to F2 along with other predefined parameters; each band 2 waveform comprises the RMS voltage of the FFT spectrum from F2 to F3 along with other predefined parameters; each band 3 waveform comprises the RMS voltage of the FFT spectrum from F3 to F4 along with other predefined parameters; and similarly, each band ‘n’ waveform comprises the RMS voltage of the FFT spectrum from Fn to Fn+1 along with other predefined parameters. In embodiments, the plurality of EEG signal frequencies corresponding to each data segment create a poly-band (for example, the segmental poly-band 210) which, in embodiments, is stored in a poly-band storage.

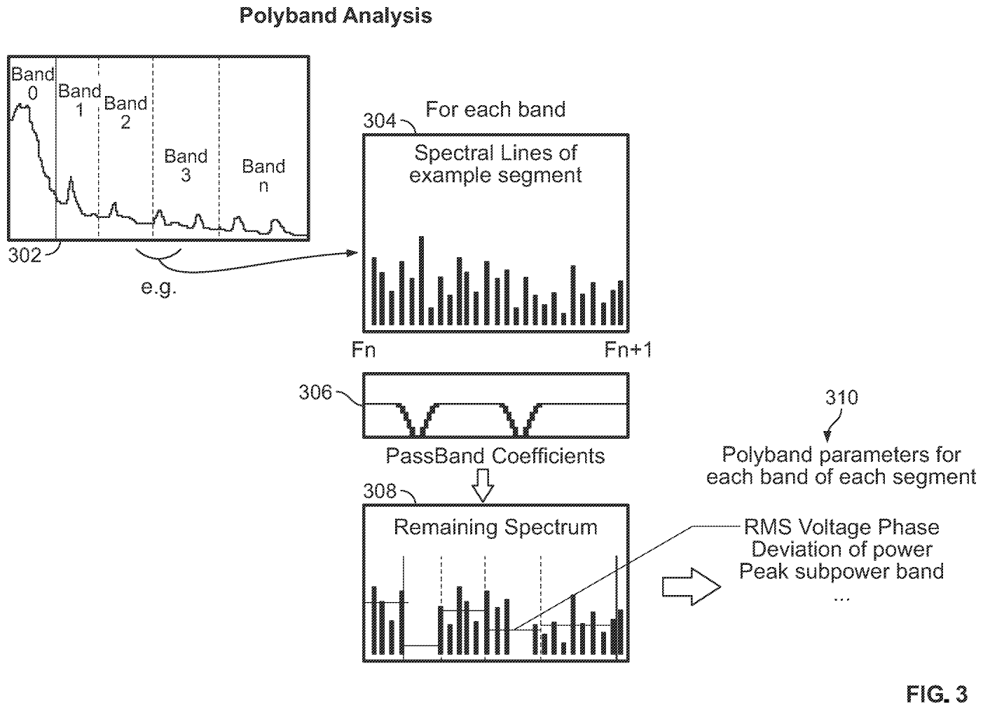

FIG. 3 is a pictorial representation of poly-band analysis of filtered EEG signals shown in FIG. 2. Graph 302 depicts an EEG data segment on which FFT is applied to obtain the filtered signals grouped in predefined frequency bands labeled as ‘Band 0’ to ‘Band n’. Graph 304 depicts the frequencies contained in each band shown in graph 302. The frequencies in each band range from F1 to Fn+1, as shown in graph 304. After the signal frequencies in each band are passed through a predefined filter characterized by predefined passband coefficients depicted in graph 306, the remaining frequency samples are depicted in graph 308. In an embodiment the passband is flat (gain=0 dB) with attenuation around 60 Hz (for example, 58 Hz to 62 Hz has a gain of −40 dB) and around each of its harmonics. The remaining frequency samples depict the EEG signals having a measurable total power, phase, deviation of power, and peak sub-power bands. In embodiments, a collection of parameters 310 for all frequency bands (only one band is shown in FIG. 3) comprises the EEG data polyband. In an embodiment, data sampling of obtained EEG signals is performed on 100 msec data segments and is analyzed to generate a predefined number (‘n’) of spectral lines. In an embodiment, a passband filter is applied to the generated spectral lines to improve Signal to Noise ratio (SNR) and the filtered spectral lines are clustered into a plurality of frequency bands. Each band is analyzed for parameters such as, but not limited to, RMS voltage. A polyband, comprising multiple frequency bands, each with multiple parameters, may be used to store the characteristics of an EEG data waveform segment without having to store the entire EEG waveform. In an exemplary case, where the number of bands is five, the data rate per channel is 5 bands per segment multiplied by 10 segments per second which is equal to 50 parameters per second; the compressed data amounts to approximately 125 bytes/second. Hence, excluding high sample rate data, the net data rate of the compressed EEG data and the polyband descriptors/parameters is approximately 175 bytes/second per channel, or approximately 35 kbytes/second for 200 channels. In an embodiment, the data rate during clinician review is approximately 100 times greater, which translates to approximately 3.5 mbytes/second, while the high sample rate data is approximately 40 mbytes/second.

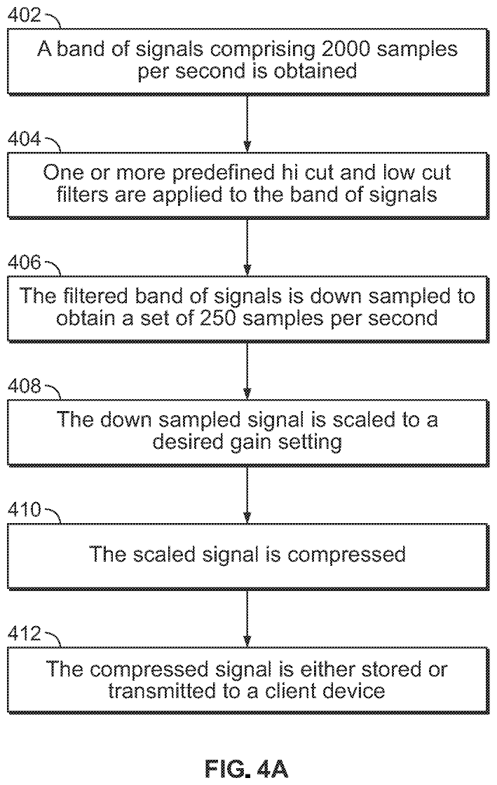

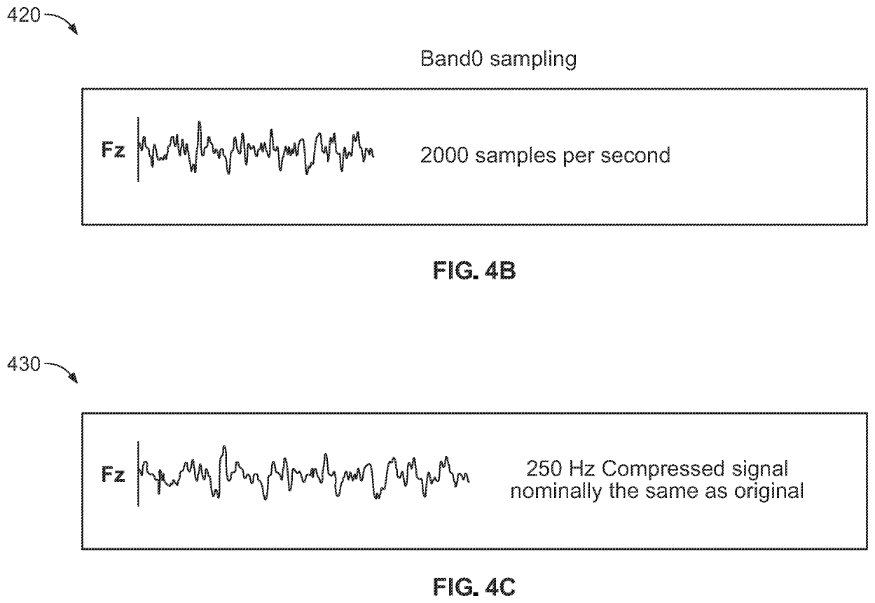

FIG. 4A is a flowchart illustrating the steps of down-sampling a band of low frequency EEG signals, in accordance with an embodiment of the present specification. FIG. 4B shows an EEG signal 420 sampled at a rate of 2000 samples per second. FIG. 4C shows the signal of FIG. 4B down-sampled and compressed to a rate of 250 samples per second, in accordance with an embodiment of the present specification. Referring to FIGS. 4A, 4B and 4C, at step 402, a band of signals comprising 2000 samples per second is obtained. At step 404, one or more predefined low cut and high cut filters (typically 2 Hz to 70 Hz) are applied to the band of signals. At step 406, the filtered band of signals is down-sampled to obtain a set of approximately 250 samples per second. At step 408, the down sampled signal is scaled to a desired gain setting. At step 410, the scaled signal is compressed. At step 412, the compressed signal is either stored or transmitted to a client device. Referring to FIGS. 4B and 4C, as can be seen, the compressed signal 430 sampled at a rate of 250 samples per second is minimally different from the original signal 420 sampled at a rate of 2000 samples per second.

In an embodiment, the system of the present specification analyzes down-sampled EEG signal portions of 100 msec duration, as HFO components of EEG signals typically occur for 100 msec durations. The HFO signals are preserved and characterized in a small number of frequency bands. In an embodiment, the system provides a 3D display of down sampled EEG signals comprising HFO signal markers superimposed upon the other/regular EEG signals. A clinician viewing the displayed EEG signals may recognize HFO signals by spotting their electrical field distribution which will differ from artifact and other biopotentials. In an embodiment, the system of the present specification enables display of high frequency signals to persist on each display window for approximately one second as the pages are updated on the display. In a situation where the update to pages is delayed or if some pages are not updated, the persistence of high frequency signals provides a notification of the signals on a subsequent page. In embodiments, the EEG signal images displayed for viewing by a clinician are generated based on the frequency bands that the signals are grouped into as described above. In an embodiment, the images comprise a legend comprising information regarding frequency, amplitude and duration of the displayed signals.

FIG. 5A illustrates a plurality of electrodes attached at different locations in a human brain for stimulating/recording brain activity with the presence of high frequency data shown in color, in accordance with an embodiment of the present specification. FIG. 5B is an EEG display obtained via the electrodes shown in FIG. 5A with the presence of high frequency data shown in color, in accordance with an embodiment of the present specification. As shown, there are 16 electrodes 502 coupled with a brain 504. Display 506 comprises waveforms 508 corresponding to brain activity recorded by the electrodes 502. As shown in FIG. 5B, frequencies of interest such as HFOs and other conventional events recorded by the electrodes 502 are marked by colored lines 510, wherein each color represents a different frequency band and the width of a colored line 510 represents the corresponding band amplitude. FIG. 5C illustrates the EEG display obtained via the electrodes shown in FIG. 5A with high frequency data shown as a frequency shifted burst in accordance with an embodiment of the present specification. As shown in FIG. 5C, the different band signals shown in FIG. 5B are frequency shifted into a range displayable on a monitor. In an embodiment, the signals are amplified relative to the low frequency components and then added to the original EEG waveform. Signal 520 depicts a high frequency burst at a high sample rate. Signal 522 depicts the signal 520 filtered and at a low sampling rate. Signal 524 depicts the signal 520 wherein the 400 Hz-600 Hz frequency components have been shifted to a 50 Hz range and have been amplified 10 times. In an embodiment, in order to obtain persistence of the frequency shifted presentation, the burst component is either redrawn over the new waveform, or the burst component is redrawn as added to the new waveform.

In an embodiment, the system of the present specification is configured to provide an acoustic indication of signals of interest such as, but not limited to, HFOs in EEG data to enable a clinician viewing the data to take note of said signals. In embodiments, the signals of interest and other event markers are correlated with a ‘sound’ which is recognizably different from background EEG and artifact signals and enables an improved detection and recognition of HFOs and seizure causing events in the EEG data. As is known, alpha waves are seen in an EEG during a normal wakeful state of a patient, while EEG delta waves are high-amplitude brain waves associated with deep sleep stages of the patient. In an embodiment, EEG data presented at 100× normal speed presents alpha waves at 1000 Hz, and delta waves at 300 Hz. These are in a good range for human hearing and discrimination. In an embodiment, in order to achieve 100× normal speed when the pagination speed may vary up or down, the acoustic data is frequency shifted back to 100× from display rates that are within 50× to 200× real time. For example, a display of 5 pages/second may be equated to an upshift of two times the normal speed, and a display of 20 pages/second may be equated to a downshift of two with respect to the normal speed. The high frequency components, lying within a range of 250 Hz to 800 Hz, when played back at 100× normal speed fall outside of the frequency range of human hearing. In an embodiment, band 1 to band ‘n’ signals are used to synthesize selectable unique sounds based on band information indicative of HFO's such as, but not limited to a pen noise, (a signature sound generated by now obsolete paper EEG machines), an acoustic chirp, or a decaying bell tone.

In an embodiment, low frequency fast paginated compressed EEG data displayed on a client device for viewing by a clinician comprises color coded/mapped high frequency components in a background. This enables the clinician to view high frequency data in low frequency modes for rapid paging as described above.

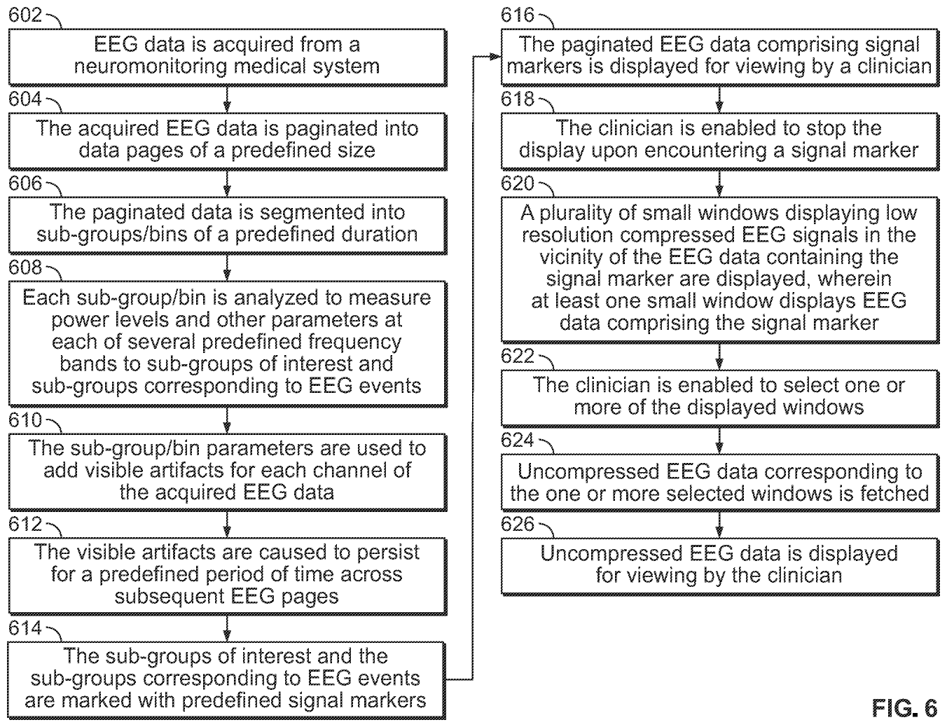

FIG. 6 is a flowchart detailing a method for acquiring and presenting high frequency EEG data, in accordance with an embodiment of the present specification. Referring simultaneously to FIG. 6 and FIG. 1A, at step 602, EEG data is acquired from a neuromonitoring medical system. In embodiments, the neuromonitoring medical system comprises a large number of electrodes, such as an EEG (electroencephalography) system, which is used for monitoring the neurological state of a patient. During use, the neuromonitoring medical device may be coupled to a patient through a plurality of electrical leads such that each of the leads is coupled to an electrode positioned at an appropriate location on the body of the patient. Each electrode is positioned at a different location to capture the electrical activity in its vicinity, and the input recorded from each electrode is collected as raw/uncompressed EEG data.

At step 604, the acquired EEG data is paginated into data pages of a predefined size (for example, 1 second pages as described above) comprising compressed EEG data. As is known pagination is a technique used to divide a data set into smaller, more manageable portions, often in the form of ‘pages’ of data that is presented one ‘page’ at a time to a client device. At step 606, the paginated EEG data is segmented into sub-groups or bins of predefined duration. In an embodiment, the paginated data is clustered/bucketed into groups by using a predefined clustering rule. In an embodiment, the paginated data is bucketed into groups of 100 msec duration. At step 608 each sub-group/bin is analyzed to measure power levels and one or more predefined parameters at each of a plurality of predefined frequency bands to determine sub-groups of interest and sub-groups corresponding to EEG events. In an embodiment, a data sub-group comprises signals of frequencies lying within a predefined range, such as, for example signals having a frequency ranging from 100 Hz to 200 Hz. In some embodiments, paginated data signals having a frequency ranging from 100 Hz to 150 Hz are clustered into one sub-group, paginated data signals having a frequency ranging from 150 Hz to 200 Hz are clustered in another sub-group, and so on such that subsequent sub-groups are defined by increasing 50 Hz ranges. In embodiments, sub-groups of interest may be sub-groups having frequencies lying within one of a plurality of predefined frequency ranges, such as, but not limited to, HFO EEG signals. In embodiments, conventional events may comprise events such as, but not limited to provision of stimulus/stimulation triggers and a patient's reaction/response to said stimulus, artifacts (eye movements, heartbeats, patient movements) or specific patterns of brain activity (epileptic spikes). In embodiments, sample rates, bin sizes, compression values, and frequency bands may be optimized for clinical EEG. In various embodiments, the system and method of the present specification also provides management and display of other physiologic and non-physiologic data whose parameters could vary widely.

At step 610, the sub-group parameters (including measured power levels) are used to add visible artifacts generated by a signal decorator 138 configured to represent frequency band specific markers for each channel of the acquired EEG data. At step 612, the visible artifacts generated by signal decorator 138 are caused to persist for a predefined period of time across subsequent EEG pages in order to enhance the presence of the artifacts. In an embodiment, the persistence time is 1 second. At step 614 the sub-groups of interest and the sub-groups corresponding to EEG events are marked with predefined signal markers. In embodiments, sub-groups of interest are marked differently than the conventional events. In an exemplary embodiment, sub-groups of interest may be marked with a predefined color/alphanumeric codes while event markers may be marked using conventionally used codes/colors. At step 616, the compressed paginated EEG data comprising signal markers is displayed for viewing by a clinician. At step 618, the clinician may stop the display, via an input, upon encountering a signal marker. At step 620, a plurality of small windows displaying low resolution compressed EEG signals in the vicinity of the EEG data containing the signal marker are displayed, wherein at least one small window displays EEG data comprising the signal marker, when the clinician stops the display of paginated EEG data. In an embodiment, a predefined number of the plurality of small windows are shown, wherein the predefined number is ‘n’. At step 622, the clinician selects one or more of the displayed windows. At step 624, uncompressed EEG data stored by the high sample rate module 112 corresponding to the one or more selected windows is fetched. At step 626 the uncompressed EEG data is displayed for viewing by the clinician.

In embodiments, threshold frequencies, meaning a high-cut frequency and a low-cut frequency for bucketing or segmenting fast data, are defined during EEG data acquisition. The data gain may, in embodiments, be changed over a range of 25 to 1. In an embodiment, data display/sweep speeds are optimized at 10 or 20 seconds per page on a display screen of the clinician. In an embodiment, as the system of the present specification is trained to recognize HFOs in the acquired EEG data, band analysis parameters are improved/changed. In some embodiments, page start and end points do not align exactly for high resolution and fast data. For example, a fast data page size of 78 blocks having 128 points is similar to a page displayed for 10 seconds. Hence, in embodiments, paginated data is bucketed/clustered in 10 second blocks. In cases where a section of EEG data is missing, the truncated fast data will have fewer chunks and upon resumption, the new fast data 0th chunk will be the first new data. In an embodiment, a transition from display of fast data to display of high-resolution data will cause beginning of display of the high resolution data at the closest second to the start of the fast data. In embodiments, fast data is compressed in such a manner so that data decompression is fast and has a lower bandwidth, thereby enhancing review performance.

The above examples are merely illustrative of the many applications of the system of present specification. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

What is claimed is:

1. A system for electroencephalogram (EEG) data acquisition and presentation comprising:
a neuromonitoring medical system configured to capture electrical activity of a patient's brain as EEG signals via a plurality of EEG electrodes;
a server coupled with the neuromonitoring medical system configured to receive and process the EEG signals wherein the server comprises:
a data compression module configured to paginate the EEG signals into a plurality of pages, wherein each of the plurality of pages is a predefined size that comprises compressed EEG data derived from the EEG signals;
a data segmentation module configured to segment the paginated, compressed EEG data into groups, wherein each group is defined by a predefined frequency range;
a data segment analysis module configured to analyze the EEG data to determine signals corresponding to EEG events; and
a client device adapted to receive and display the analyzed EEG data for viewing by a clinician, wherein the client device comprises at least one processor and programmatic instructions that, when executed by the at least one processor:
sequentially displays a plurality of windows, wherein each of the plurality of windows is adapted to display a first version of the EEG data having a first resolution and wherein each of the plurality of windows is configured to display a predefined time range of the first version of the EEG data;
receives a selection of one or more of the displayed plurality of windows;
based on the selection, fetches a second version of the EEG data, wherein the second version has a second resolution and wherein the second resolution is greater than the first resolution; and
displays the second version of the EEG data.

2. The system of claim 1, wherein the second version of the EEG data is an uncompressed version of the EEG data and wherein the first version of the EEG data is a compressed version of the EEG data.

3. The system of claim 1, wherein the data segment analysis module is configured to analyze the EEG data to determine signals of interest and to mark the signals of interest and the EEG events with signal markers.

4. The system of claim 3, wherein the data segment analysis module is configured to mark the signals of interest using a first type of predefined signal markers and mark the EEG events using a second type of predefined signal markers, wherein the first type of predefined signal markers is different from the second type of predefined signal markers.

5. The system of claim 3, wherein at least some of the signals of interest comprise high frequency oscillations (HFO).

6. The system of claim 3, wherein at least some of the signals of interest comprise one or more EEG signals having a frequency outside of predefined frequency ranges, wherein the ranges include at least 80 Hz to 250 Hz and 250 Hz to 500 Hz.

7. The system of claim 3, wherein the predefined signal markers comprise audible signal markers.

8. The system of claim 1, wherein the predefined time range displayed by each of the plurality of windows is in a range of 1 second to 5 minutes.

9. The system of claim 3, wherein each of the plurality of windows is adapted to display 1 to 120 seconds of the first version of the EEG data occurring before one of the predefined signal markers and adapted to display 1 to 120 seconds of the first version of the EEG data occurring after said one of the predefined signal markers.

10. The system of claim 1, wherein the server further comprises a cache memory and wherein the cache memory is configured to store the segmented, paginated, compressed EEG data.

11. The system of claim 10, wherein the server further comprises a cache controller coupled with the cache memory and wherein the cache controller is configured to control a transmission of the segmented, paginated, compressed EEG data stored in the cache memory to the client device.

12. The system of claim 10, wherein the cache memory is adapted to store the second version of the EEG data corresponding to the selected one of the plurality of windows.

13. The system of claim 1, wherein the client device further comprises programmatic instructions that, when executed by the at least one processor, generates a three dimensional display of the first version of the EEG data and wherein the three dimensional display comprises signal markers visually indicating where high frequency oscillations occur in the EEG signals.

14. The system of claim 3, wherein the client device further comprises programmatic instructions that, when executed by the at least one processor, enables a user to stop a display of the first version of the EEG data upon encountering one or more of the signal markers.

15. The system of claim 1, wherein a first group of compressed EEG data is defined by a frequency ranging 100 Hz to 150 Hz, a second group of compressed EEG data is defined by a frequency ranging from 150 Hz to 200 Hz, and subsequent groups of compressed EEG data are defined by increasing 50 Hz ranges.

16. A method of processing and presenting EEG data comprising:
acquiring EEG data;
paginating the EEG data into a plurality of pages, wherein each of the plurality of pages has a predefined size and comprises the EEG data in a compressed format;
grouping the paginated, compressed EEG data into one or more frequency bins;
analyzing the grouped, paginated, compressed EEG data to determine at least one of signals of interest or signals corresponding to EEG events;
marking at least one of the signals of interest or the signals corresponding to EEG events with signal markers;
displaying the grouped, paginated, compressed EEG data comprising the signal markers by:
enabling a user to stop the display upon encountering at least one of the signal markers;
displaying a plurality of windows, wherein each of the plurality of windows is adapted to display a low-resolution version of the EEG signals in a vicinity of the grouped, paginated, compressed EEG data containing the signal marker;
enabling the user to select at least one of the plurality of windows; and
fetching a high-resolution version of the EEG data in the selected at least one of the plurality of windows; and
displaying the fetched high-resolution EEG data.

17. The method of claim 16, wherein the signal markers comprise a first type and a second type, wherein the first type of signal marker is used to mark signals of interest and wherein the second type of signal marker is used to mark signals corresponding to EEG events.

18. The method of claim 16, wherein a signal of interest comprises high frequency oscillations (HFO).

19. The method of claim 16, wherein a signal of interest comprises one or more EEG signals having a frequency outside of predefined frequency ranges, wherein the ranges include at least 80 Hz to 250 Hz and 250 Hz to 500 Hz.

20. The method of claim 16, wherein the signal markers comprise audible signal markers.

21. The method of claim 16, further comprising displaying three dimensional compressed EEG signals comprising high frequency oscillations signal markers superimposed upon other EEG signals.

22. The method of claim 16, wherein grouping the paginated EEG data into frequency bins comprises groups of EEG signals having a time resolution of approximately 100 msec and having bandwidth spreads in a range of 50 Hz to 100 Hz.

23. The method of claim 16, wherein each of the EEG events comprises one or more of a stimulus trigger, an EEG response to a stimulus, artifacts of brain activity, and specific patterns of brain activity.

24. The method of claim 16, wherein displaying the grouped, compressed, paginated EEG data comprises displaying the grouped, compressed, paginated EEG data at a rate ranging from 1 to 100 pages per second.

25. The method of claim 16, wherein a first bin of compressed EEG data is defined by a frequency ranging 100 Hz to 150 Hz, a second bin of compressed EEG data is defined by a frequency ranging from 150 Hz to 200 Hz, and subsequent bins of compressed EEG data are defined by increasing 50 Hz ranges.

26. A system for electroencephalogram (EEG) data acquisition and presentation comprising:
an EEG data acquisition system configured to capture an electrical activity of a patient's brain as EEG signals via a plurality of EEG electrodes;
a server coupled with the neuromonitoring medical system configured to receive and process the EEG signals wherein the server comprises:
a data compression module configured to paginate the EEG signals into a plurality of pages, wherein each of the plurality of pages comprises compressed EEG data derived from the EEG signals;
a data segmentation module configured to segment the paginated, compressed EEG data into groups, wherein each group is defined by a predefined frequency range;
a data segment analysis module configured to analyze the EEG data to determine data corresponding to EEG events; and
a client device adapted to receive and display the compressed and analyzed EEG data for viewing by a clinician, wherein the client device comprises at least one processor and programmatic instructions that, when executed by the at least one processor:
sequentially displays a plurality of windows, wherein each of the plurality of windows is adapted to display a first version of the EEG data having a first resolution and display one or more first parameters and wherein each of the plurality of windows is configured to display a predefined time range of the first version of the EEG data;
receives a first selection of one or more of the displayed plurality of windows;
based on the first selection, fetches a second version of the EEG data, wherein the second version has a second resolution and is associated with one or more second parameters and wherein the second resolution is greater than the first resolution and at least one of the second parameters is different from at least one of the first parameters;
displays the second version of the EEG data;
receives a second selection of the one or more of the displayed plurality of windows chosen by the first selection;
based on the second selection, fetches a third version of the EEG data, wherein the third version has the second resolution and is associated with at least one of the first parameters; and
displays the third version of the EEG data.

27. The system of claim 26, wherein the first parameters and the second parameters comprise at least one of high cut filter settings, low cut filter settings, gain, sweep, or page position.

28. The system of claim 26, wherein the client device further comprises programmatic instructions that, when executed, generates a displayable navigation bar having a first portion and a second portion and that is adapted to cause the first selection when the first portion is activated.

29. The system of claim 28, wherein the displayable navigation bar is further adapted to cause the selection when the second portion is activated.

30. The system of claim 26, wherein the client device further comprises programmatic instructions that, when executed by the at least one processor, generates a three dimensional display of the compressed and analyzed EEG data and wherein the three dimensional display comprises signal markers visually indicating where high frequency oscillations occur in the EEG signals.

31. The system of claim 26, wherein the client device further comprises programmatic instructions that, when executed by the at least one processor, are configured to stop a display of the compressed and analyzed EEG data upon encountering one or more of the signal markers.
